(19) United States
(12) Patent Application Publication
(10) Pub. No.: US 2003/0215486 A1
(43) Pub. Date: Nov. 20, 2003

(54) Cleansing or moisturizing mitt or glove

(76) Inventors: Craig J. Berry, Westport, CT (US); Kim R. Berry, Westport, CT (US)

Correspondence Address: PERMAN & GREEN, 425 POST ROAD, FAIRFIELD, CT, 06824, US

(21) Appl. No.: 10/146,783

(22) Filed: May 15, 2002

(51) Int. Cl. A61K 9/70

(52) U.S. Cl. 424/443

(57) ABSTRACT

A cleansing article comprising a first substrate coated with at least one cosmetic formulation, and a second substrate coated with at least one cosmetic formulation. The first and second substrates are adapted to be joined together along corresponding regions of each of the first and second substrates to form a cavity between the first and second substrates. The cavity has an opening into which a band can be inserted and the resulting article is substantially dry and adapted to be wetted for use.

### BACKGROUND OF THE INVENTION

1. Field of the Invention

The present invention generally relates to coated, non-woven articles, and in particular to a personal cleansing article.

2. Brief Description of Related Developments

Personal cleansing products come in a variety of different shapes and forms. Generally, cosmetic and over the counter cleansing or conditioning products can include bar soaps, creams, lotions and gels. Uses of these products can include facial, body and skin treatments.

Problems with the various forms of personal cleansing products include the balancing of cleansing efficiency versus delivering a conditioning benefit. One solution to this problem is to use separate cleansing and conditioning products. However, this is not always convenient or practical and many consumers would prefer to use a single product, which can both cleanse and condition the skin or hair. In a typical cleansing composition the conditioning ingredients are difficult to formulate because many conditioners are incompatible with the surfactants, resulting in an undesirable non-homogenous mixture. To obtain a homogeneous mixture with conditioning ingredients, and to prevent the loss of conditioning ingredients before deposition, additional ingredients, e.g. emulsifiers, thickeners, and gellants are often added to suspend the conditioning ingredients within the surfactant mixture. This results in an aesthetically pleasing homogenous mixture, but often results in poor deposition of conditioning ingredients, because the conditioners are emulsified and not efficiently released during cleansing. Also, many conditioning agents have the disadvantage of suppressing lather generation. Lather suppression is a problem because many consumers seek cleansing products that provide a rich, creamy, and generous lather.

### SUMMARY OF THE INVENTION

The present invention is directed to a cleansing article. In one embodiment, the cleansing article comprises a first substrate coated with at least one cosmetic formulation and a second substrate coated with at least one cosmetic formulation. The first and second substrates are adapted to be joined together along corresponding regions of each of the first and second substrates to form a cavity between the first and second substrates. The cavity has an opening into which a hand can be inserted. The resulting article is substantially dry and adapted to be wetted for use. In one aspect, the present invention is directed to a cleansing mitt. In one embodiment the cleansing mitt comprises a pair of layers of a non-woven material that are coated with one or more cosmetic formulations. A heat bond is formed along at least a perimeter of each layer, and the pair of layers are joined together along at least three sides of the pair of layers to form an opening along one side. The opening is adapted to allow a user to insert a body extremity into the opening. The pair of layers are substantially dry and adapted to be wetted for use.

### BRIEF DESCRIPTION OF THE DRAWINGS

The foregoing aspects and other features of the present invention are explained in the following description, taken in connection with the accompanying drawings, wherein:

FIG. 1 is a perspective view of one embodiment of a coated, non-woven article incorporating features of the present invention.

FIG. 2 is a perspective view of one embodiment of a shaped article incorporating features of the present invention.

FIG. 3 is a partial schematic view of one embodiment of the coating portion of an apparatus incorporating features of the present invention.

FIG. 4 is a partial schematic view of the drying, bonding and cutting portions of an apparatus incorporating features of the present invention.

FIG. 5 is a partial schematic view of one embodiment of a striping portion of an apparatus incorporating features of the present invention.

### DETAILED DESCRIPTION OF THE PREFERRED EMBODIMENTS

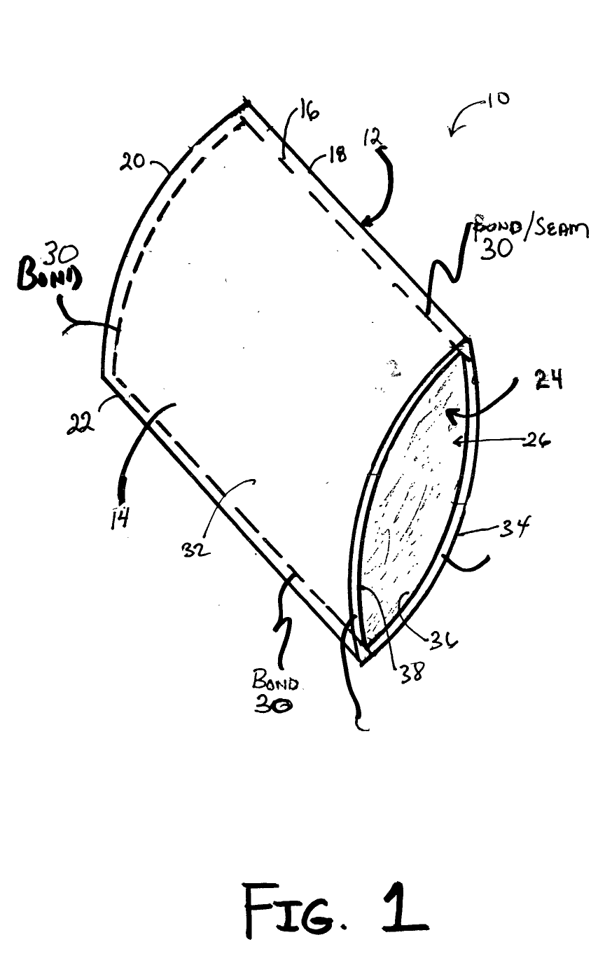

Referring to FIG. 1, one embodiment of a coated, non-woven article 10 incorporating features of the present invention is illustrated. Although the present invention will be described with reference to the embodiment shown in the drawings, it should be understood that the present invention could be embodied in many alternate forms of embodiments. In addition, any suitable size, shape or type of elements or materials could be used.

As shown in FIG. 1, the article 10 generally comprises two layers 12, 14 of a material joined together along an outer perimeter 16 of each layer. The material generally comprises a non-woven cloth, although other materials that are adapted to be coated with a slurry can be used. As shown in FIG. 1, the two layers 12, 14 can joined together along sides 18, 20 and 22 to leave an opening 24 along the unjoined side 26. Alternatively, any suitable number of sides can be joined.

Generally, each layer 12, 14 comprises a non-woven material that is adapted to be coated. In alternate embodiments, each layer can comprised any suitable material can be used that can be coated with any one of a number of body cleansing or conditioning agents or materials which can then be applied to parts of the body. It is a feature of the present invention to use the articles to apply various skin cleansing or conditioning agents to the body.

Each layer 12, 14 of the article 10 generally comprises a non-woven, cloth like material. In one embodiment, each layer 12, 14 comprises a water insoluble substrate and can include a wet extensible portion. Each layer 12, 14 is generally adapted to be coated with a suitable cosmetic or over the counter body cleansing or skin conditioning agents, ingredients or materials.

The present invention is generally adapted to incorporate a user's favorite cosmetic or over the counter (“OTC”) formulations in a dry, inactive state, that is activated when a liquid such as water is added to the article 10. In this manner, the article 10 can be sized, packaged and adapted for ease of use in a variety of different applications, such as for example while traveling. The article 10 would be generally dry while stored, and when removed for use, wetted to activate the formulations in the materials or layers 12, 14 to achieve the desired result. This could include cleansing, bathing or showering. The article 10 could also comprise a single use, disposable article. The incorporation of the cosmetic or OTC formulation into the non-woven material can generally enhance the performance of the formulation.

Examples of some of the formulations or applications for the present invention can include, but are not limited to, facial, body and skin treatments and products, bath and shower gels, conditioners, cleansers, shampoos, facial treatments, acne treatments, eye and face make-up removers, and face and body creams and lotions. As used herein, the term “cosmetic” generally refers to examples of the foregoing formulations and products.

Although the layers 12, 14 of the article 10 are generally described as comprising the same material and carrying the same formulations and products, in alternate embodiments, the two layers 12, 14, or sublayers thereof, could comprise different materials and each could carry different formulations or products or combinations thereof.

As shown in FIG. 1, the article 10 is formed by sealing the two layers 12, 14 of material around at least a portion of the perimeter 16. As shown in FIG. 1, the article 10 is bonded along sides 18, 20 and 22. The article 10 can comprise two layers 12, 14 of a heat or sonically bondable material. In alternate embodiments, any suitable means can be used to bond the two layers, including for example stiching or gluing. It is a feature of the present invention that the two layers remain bonded together even when the article is wetted or submersed in water. In one embodiment, the layers 12 and 14 comprise a material or materials that when heated and melted bonds under a certain degree of pressure to itself. Examples of such materials can include polypropylene.

In an embodiment where the layers 12, 14 include one or more sub-layers, at least the outermost portions of each layer 12, 14 that make contact must have heat bondable properties in order to form a desired bond 30 around portions of the perimeter 16 of the article. Examples of such heat bondable materials or substrates can include, but are not limited to, Rayon and Polypropylene, and Rayon and Polyester. The material can be of any desired thickness and texture in order to accommodate the cosmetic formulation and still be pleasing to the user. The article 10 could include different textures on each outer side 32, 34 of the article 10, or on the interior sides 36, 38 of the article 10.

Referring to FIG. 3, one embodiment of a method for coating each layer 12, 14 of the article 10 is illustrated.

As shown in FIG. 3 the article 10 can be formed by first coating layer 52 of non-woven material taken from a first roll 50 by means of, for example, a knife-over-roll coater 56 with the wet cosmetic or OTC formulation 60. In alternate embodiments, a suitable method of laying down a product can be used, such as for example a slot die, a reverse roll, or a gravure roll. The formulation 60 can be fed or pumped to the knife-over-roll coater 56, using for example a pump 62 and tubing 64. In alternate embodiments, any suitable or desired material adding or coating process can be used, such as for example an impregnating process. It is a feature of the present invention to be able to add any desired cosmetic or cleansing formulation to the layer 52. A second layer 72 taken from a second roll 70 of a non-woven material is also coated using the same or similar coating process.

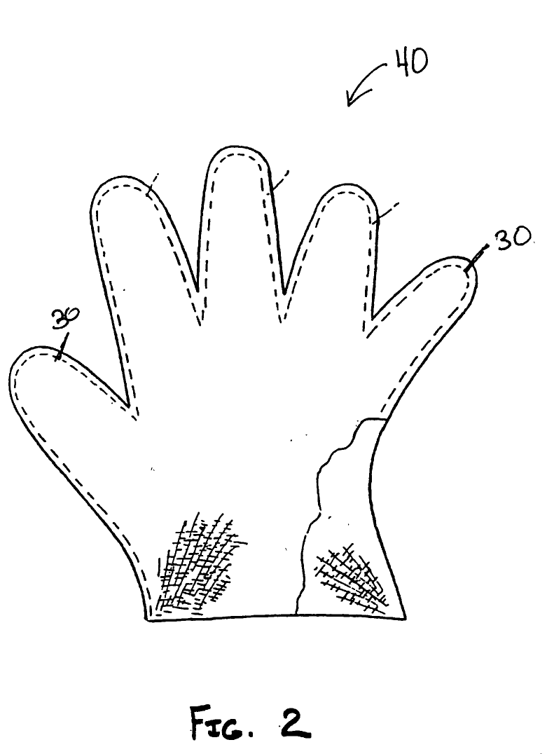

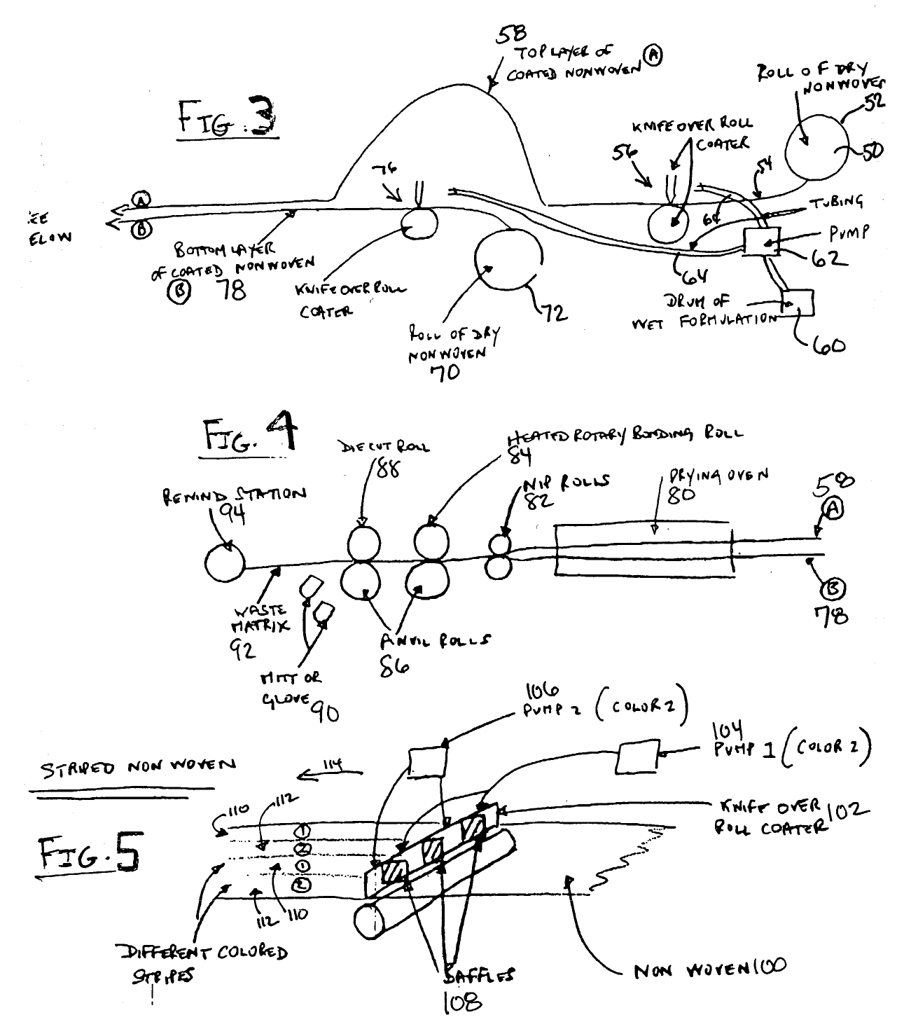

Referring to FIG. 4, after coating, both layers 58, 78 of the coated material can be passed through a drying oven 80 in order to dry water or solvent off the wet formulation 60. The layers 58, 60 are kept in the oven 80 for a period of time sufficient for the two layers 58, 78 to be dry to the touch upon exiting the drying oven 80. After drying, the two layers 58, 78 can be brought together by nip rolls 82 and then passed through a heated rotary die 84 and anvil roll 86 which compresses and heat seals the two layers 58, 78. The two layers 58 and 78 can be sealed into the outline of the desired shape, such as for example a mitt or glove that can be fitted over a hand. One example of such a mitt shape is shown in FIG. 2. Although the present invention is described as a mitt or a glove shape, it should be understood that any desired shape could be formed, such as for example, a circle, oval, square, or rectangle. A feature of the present invention is that the article can be placed over the hand and used in the bath or shower is a manner similar to a washcloth, except that the article stays in position over the hand and does not necessarily need to be held or gripped in order to be used. For example, the article could be configured to be in the shape of a cartoon figure, a sports action figure, a flower, or any other user pleasing shape. The shape can be formed in any suitable manner, such as for example a die cut to form a distinct shape. The sealed shape 90 can then be die cut by a die cut roll 85 around the perimeter of the bonded shape and removed from the waste matrix 92. In alternate embodiments, it may not be desireable to seal the two layers along the entirety of the perimeter. For example, it may be desirable to leave spaces in the perimeter along one or more sides to allow a user to insert their fingers between the layers with the bulk of the article being positioned in the palm area of the hand. The fingers could thus extend out of the end of the article from which the hand entered, while still being held securely over the palm to enable using the article similar to a washcloth or cleansing bar. The formulation could also include a surfactant.

In one embodiment, referring to FIG. 5, different portions or sections 110, 112, of the layer 100 of non-woven material, can be coated with different formulations. For example, in one embodiment, sections 110, 112 could be coated with a formulation or product in the form of strips along a direction 114 of travel of the layers 100.

Each section 110, 112 could include a different formulation or product. A different color could be used in each section or “stripe” 110, 112 to distinguish between the different formulations. In alternate embodiments, only selected portions of the article or material 110 might be coated with the cosmetic formulation. Also, different sections of each layer could include different formulations that achieve a different effect on the skin or body.

Although only two sections 110, 112 are shown, it should be understood that any suitable number of sections or stripes could be used.

Each formulation can be pumped into the channels 110, 112 in any suitable fashion prior to passing the material through the knife-over-roll coater 102. For example, pumps 104, 106 could be used to apply the different formulations and/or colors, through baffles 108 to the material 100 as it passes through the knife over roll coater 102. In alternate embodiments, any suitable number of pumps can be used to apply a corresponding number of stripes or sections 110, 112 of formulation. A similar process can be used to apply the mixed coating to the other layer. It should also be understood that in alternative embodiments, different formulations or colors could be applied to each layer of the two layers that form the article 10 of FIG. 1. For example, referring to FIG. 1, one layer 12 could be coated with a first formulation and the second layer 14 coated with a second formulation. The first and second formulation could each be colored differently in order for the user to be able to distinguish between the different formulations on each layer. Again, the first layer 12 could include a number of different formulations while the second layers 14 also includes a number of different formulations. The formulations could all be different or some may be the same.

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

What is claimed is:

1. A cleansing article comprising:
a first substrate coated with at least one cosmetic formulation; and
a second substrate coated with at least one cosmetic formulation, the first and second substrates being adapted to be joined together along corresponding regions of each of the first and second substrates to form a cavity between the first and second substrates, the cavity having an opening into which a hand can be inserted; and
wherein the resulting article is substantially dry and adapted to be wetted for use.

2. The cleansing article of claim 1 wherein the first and second substrate each comprise a non-woven cloth-like material.

3. The cleansing article of claim 1 wherein the first and second substrate each include a water insoluble substrate and a wet extensible portion.

4. The cleansing article of claim 1 wherein the article is a single use, disposable article.

5. The cleansing article of claim 1 wherein the article is formed in a shape of a glove which is adapted to fit over a hand.

6. The cleansing article of claim 1 wherein an outer perimeter of each of the first substrate and the second substrate is heat bondable, and wherein when the first substrate and the second substrate are formed and heat is applied, the perimeter of the first substrate and the perimeter of the second substrate are heat bonded.

7. The cleansing article of claim 1 wherein the first substrate is coated with a first cosmetic formulation that is different from a second formulation used to coat the second substrate.

8. The cleansing article of claim 1 wherein the first substrate is divided into sections and a different formulation is coated onto at least one of the sections.

9. The cleansing article of claim 1 wherein the first substrate is coated with at least two cosmetic formulations, each formulation being coated on the substrate adjacent to another formulation.

10. The cleansing article of claim 9, wherein each formulation is coated on the substrate in a form of a stripe, each stripe representing a different formulation.

11. The cleansing article of claim 9, wherein each stripe has a different color.

12. The cleansing article of claim 1 wherein both the first substrate and the second substrate are coated with a respective series of cosmetic formulations, each formulation being coated in a form of a stripe along the first substrate and the second substrate, wherein one formulation is coated adjacent to another substrate.

13. The cleansing article of claim 1 wherein the first substrate and the second substrate comprise a heat-bondable material, and the first substrate and the second substrate are heat-bonded together.

14. The cleansing article of claim 1 wherein the first substrate and the second substrate are adapted to be sonically bondable together.

15. The cleansing article of claim 1 wherein each of the first substrate and the second substrate include a bonding material that is adapted to be heat bondable and sonically bondable on itself.

16. The cleansing article of claim 15 wherein the bondable material is polypropylene.

17. A cleansing mitt comprising:
a pair of layers of a non-woven material that are coated with one or more cosmetic formulations;
a heat bond along at least a perimeter of each layer, wherein the pair of layers are joined together along at least three sides of the pair of layers to form an opening along one side, the opening adapted to allow a user to insert a body extremity into the opening; and
wherein the pair of layers are substantially dry and adapted to be wetted for use.
